(19) United States
(12) Patent Application Publication
(10) Pub. No.: US 2006/0100468 A1
(43) Pub. Date: May 11, 2006

(54) Method for hydrogenating olefin

(75) Inventors: Noriaki Oku, Ichihara-shi (JP); Masaaki Katao, Ichihara-shi (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED, Chuo-ku, Tokyo (JP)

(21) Appl. No.: 10/541,848

(22) PCT Filed: Dec. 18, 2003
(86) PCT No.: PCT/JP03/16212

§ 371 Date: Jul. 12, 2005

(30) Foreign Application Priority Data
Jan. 16, 2003 (JP) 2003-008049

(51) Int. Cl. C07C 5/03

(52) U.S. Cl. 585/250

(57) ABSTRACT

A process for hydrogenating an olefin, which comprises upwardly passing a liquid containing an olef in and a gas containing hydrogen through a packed bed of a solid hydrogenation catalyst, wherein the superficial velocity of the gas is 3.0 cm/sec or more.

### TECHNICAL FIELD

The present invention relates to a process for hydrogenating an olefin.

### BACKGROUND ART

As a process for hydrogenating an olefin, by which a liquid containing an olefin and a gas containing hydrogen are passed upwardly through a packed bed of a solid hydrogenation catalyst, for example, U.S. Pat. No. 3,127,452 discloses a technique in which α-methyl styrene and hydrogen are supplied to a catalyst bed under an upward flow to carry out hydrogenation. However, conventional processes were not necessarily satisfied from the view point of production of cumene at low cost.

### DISCLOSURE OF THE INVENTION

The present invention provides a process for hydrogenating an olefin, having an excellent effect on which the reaction rate per unit of a catalyst is high.

That is, the present invention relates to a process for hydrogenating an olefin, which comprises passing upwardly a liquid containing an olefin and a gas containing hydrogen through a packed bed of a solid hydrogenation catalyst, wherein the superficial velocity of the gas is 3.0 cm/sec or more.

### MODE FOR CARRYING OUT THE INVENTION

As a solid hydrogenation catalyst used for hydrogenation of an olefin, for example, a metal oxide containing at least one of CuO, Cr₂O₃, ZnO, FeO₃, Al₂O₃, La₂O₃, Sm₂O₃, CeO₂, ZrO₂, TiO₂, SiO₂, MnO₂, Co₂O₃, NiO, BaO, CaO and MgO; and a noble metal catalyst containing Pd, Rh, Pt or Ru, can be listed. As the solid hydrogenation catalyst, a carrier may be used or not. As the carrier, there can be listed metal oxides or compound oxides thereof such as silica, alumina, titania, zirconia, magnesia and silica-alumina; and bentonite, montmorillonite, diatomaceous earth, acidic white clay, active carbon, ceramics and the like.

In addition, as a shape of the catalyst, sphere and cylindrical form are illustrated. The catalyst is usually 0.5 to 10 mm in size, and when the size is too small, a pressure loss increases and leads to uneconomical, on the other hand, when too large, it is not preferable because a catalyst activity decreases and a reaction fluid flows non-uniformly.

The hydrogenation of an olef in is carried out using areactor in which the solid hydrogenation catalyst is packed. The reaction temperature is usually 20 to 500° C., preferably 40 to 350° C., and the reaction pressure is usually 0.1 to 20 MPa, preferably 0.1 to 10 MPa.

The used amount of the catalyst is 0.01 to 50 hr⁻¹ in terms of a space velocity of the liquid containing the olef in supplied, and a liquid containing a fresh olefin as a raw material may be diluted with an appropriate solvent or with a liquid obtained by recycling a part of the reaction mixture liquid after hydrogenation. The amount of hydrogen is usually 1.0 to 30 times by mole per an olefin to be supplied, further, hydrogen may be diluted with a gas which does not react with the olef in supplied, and extra hydrogen may be recycled.

As the olefin, compounds having a double bond or triple bond such as a styrene can be listed. The styrene includes styrene, α-methyl styrene and the like.

In the present invention, a liquid containing an olefin and a gas containing hydrogen are passed upwardly (upflow) through apacked bed of a solid hydrogenation catalyst. As reasons why the upflow is applied, a runaway of the reaction according to difficulty of control of the reaction temperature caused by generation of hot spots, can be suppressed because the liquid can be uniformly flowed in the packed bed without localization.

The utmost characteristic of the present invention is to control a superficial velocity of the gas to 3.0 cm/sec or higher, preferably 3.5 cm/sec or higher. When the superficial velocity of the gas is lower than 3.0 cm/sec, the apparent reaction rate lowers, namely the reacted amount per unit of the packed bed lowers because a rate of which hydrogen dissolves in the liquid, controls, due to a fast rate of olefin hydrogenation, the rate of the reaction. In addition, the lowering of the hydrogenation rate of the olefin leads to lowering of the yield through a tar formation reaction caused by formation of an olefin dimer and olef in polymer. The superficial velocity of the gas is preferably 10 cm/sec or lower. When the superficial velocity of the gas is higher than 10 cm/sec, a pressure loss of the packed bed may increase because wear and tear powder of the catalyst is formed by friction among catalysts.

The superficial velocity of a gas is determined by the following equation (1):
(Superficial velocity of gas)=(Real volume velocity of gas)/(Cross-sectional area of reactor) (1)

### EXAMPLE

Next, the present invention is explained by Examples.

### Example 1

A catalyst (12 cc) containing copper was packed into a reactor having an inner diameter of 4 mm, a liquid containing α-methyl styrene (AMS) of 21% by weight was supplied at a rate of 48 g/min and hydrogen of 1.5 times by mole per mole of AMS fed was fed to the reactor. The reaction was carried out under a pressure of 1.0 MPa at a temperature of 200° C. At this time, the superficial velocity of the gas was 7 cm/sec, and the reaction amount of α-Methyl styrene per catalyst was 49 kmol/m³ catalyst/hr.

### Example 2

A catalyst (2.2 cc) containing palladium was packed into a reactor having an inner diameter of 4 mm, a liquid containing α-Methyl styrene (AMS) of 18% by weight was fed at a rate of 3.3 g/min and hydrogen of 1.5 times by mole per mole of AMS fed was fed to the reactor. The reaction was carried out under a pressure of 1.0 MPa at a temperature of 180° C. At this time, the superficial velocity of gas was 6.5 cm/sec, and the reaction amount of α-Methyl styrene per catalyst was 71 kmol/m3 catalyst/hr.

### Comparative Example 1

This experiment was carried out under the same conditions as in Example 1 except that the raw material was fed at a rate of 1.6 g/min and the reaction was carried out at a temperature of 210° C. under a pressure of 1.4 MPa. At this time, the superficial velocity of gas was 2.7 cm/sec, and the reaction amount of α-Methyl styrene per catalyst was 14 kmol/m³ catalyst/hr.

### Comparative Example 2

This experiment was carried out under the same conditions as in Example 2 except that the raw material was fed at a rate of 1.6 g/min. At this time, the superficial velocity of gas was 2.8 cm/sec, and the reaction amount of α-Methyl styrene per catalyst was 42 kmol/m³ catalyst/hr.

### INDUSTRIAL APPLICABILITY

As described above, according to the present invention, there is provided a process for hydrogenating an olef in, under which a liquid containing the olefin and a gas containing hydrogen are passed upwardly through a packed bed of a solid hydrogenation catalyst, wherein the process has an excellent effect that the reaction rate per unit catalyst is high.

1. A process for hydrogenating an olefin, which comprises passing upwardly a liquid containing an olefin and a gas containing hydrogen through a packed bed of a solid hydrogenation catalyst, wherein the superficial velocity of the gas is 3.0 cm/sec or more.

2. The process according to claim 1, wherein the olef in is a styrene.

3. The process according to claim 1 or 2, wherein the superficial velocity of the gas is 3.5 to 10 cm/sec.
